(19) United States
(12) Patent Application Publication
(10) Pub. No.: US 2025/0006325 A1
(43) Pub. Date: Jan. 2, 2025

(54) FACILITATING DIGITAL REFERRALS

(71) Applicant: Bright Referral LLC, Minneapolis, MN (US)

(72) Inventors: ELIZABETH SUDIT, Minneapolis, MN (US); GEOFFREY SUDIT, Minneapolis, MN (US); CHRISTOPHER HENTGES, Crystal, MN (US)

(73) Assignee: Bright Referral LLC, Minneapolis, MN (US)

(21) Appl. No.: 18/754,641

(22) Filed: Jun. 26, 2024

Related U.S. Application Data
(60) Provisional application No. 63/510,804, filed on Jun. 28, 2023.

(51) Int. Cl. G16H 10/65; G06F 16/955

(52) U.S. Cl. CPC G16H 10/65; G06F 16/955

(57) ABSTRACT

A method, system, and platform for facilitating referrals is disclosed. The referral facilitation may include synching a smart card with a referral recipient account and a referral source. The referral facilitation further includes tapping or scanning the smart card by using a mobile device to retrieve a web address associated with the referral platform and an identifier associated with one or more of the referral recipient account and the referral source. In some embodiments, referrals are facilitated without use of a smart card. The referral facilitation further includes providing a referral form to the mobile device and receiving referral data from the mobile device. In some embodiments, the referral data includes text and audio data that may be provided to a referral recipient.

### CROSS-REFERENCE TO RELATED APPLICATIONS

The present application claims priority from U.S. Provisional Patent Application No. 63/510,804, filed on Jun. 28, 2023, the disclosure of which is hereby incorporated in its entirety.

### BACKGROUND

In the healthcare industry, the referral process may be cumbersome and lead to suboptimal outcomes for all parties. The referrer (e.g., a general practitioner) must accurately remember information of the referral recipient (e.g., a specialist) and accurately convey that information to an individual who is being referred (e.g., a patient). The individual must accurately record the information and use that information to follow up with the referral recipient. The referral recipient must be ready to receive and respond to the individual in the communication medium selected by the individual. If any of these steps break down, then the referral process may fail. As a result, an individual may not receive the requisite care, the referrer may fail to connect the individual with the requisite care, and a referral recipient may not be aware of a potential referral at the outset. Furthermore, even if a referral is successful, the referral process may, in some instances, be suboptimal, as it may be slow, inefficient, and inconsistent.

### SUMMARY

Aspects of the present disclosure relate to a platform that facilitates the making, receiving, and tracking of referrals. Although aspects of the present disclosure are described in connection with the use of smart cards to facilitate referrals, the present disclosure is not limited to embodiments in which smart cards are used. For example, a referral source may provide a link to a referral platform associated with a referral recipient by using other technology that may encode or otherwise include a web address.

In example aspects, a referral recipient, such as a healthcare specialist, may create an account with a referral platform. As part of configuring an account, the referral recipient may create profiles for one or more referral sources, which may be people or offices that refer individuals to the referral recipient. For each referral source, the referral recipient may customize a form that is shown to individuals that are referred by that referral source.

In example aspects, the referral recipient may use the referral platform to order smart cards, which may include a near field communication (NFC) chip or quick response (QR) code. Once the referral recipient receives the smart cards, the referral recipient may, for each smart card, synch the smart card with a particular referral source, thereby linking the smart card with the referral recipient, a particular referral source, and the form that the referral recipient customized for individuals associated with the referral source (e.g., patients). The referral recipient may dynamically allocate smart cards, synching some smart cards with one referral source and some smart cards with another referral source.

In example aspects, the referral recipient may distribute the smart cards to the referral sources for which the smart cards are synched. As a result, each referral source may have one or more smart cards received from the referral recipient that may be used to refer patients to the referral recipient. When a referral source refers an individual, the referral source may provide a smart card to the individual and request the individual to use a mobile device to tap or scan the card, thereby retrieving data from an NFC chip or QR card on the smart card. Based on the data retrieved from the smart card, the individual's mobile device may be configured to open a web browser and access a webpage of the referral platform.

In example aspects including medical referrals, the referral platform may, based on the data received from the patient's mobile device, determine a customized referral form that is associated with the referral recipient and the referral source, and the referral platform may provide the referral form to the patient. The patient may then use the mobile device to complete the form. For example, one or more of the patients or a referrer may input referral data into the referral form. In examples, the patient may also follow one or more customized links in the referral form that lead to websites specified by the referral recipient, such as a link to an online scheduling system, insurance information, or additional data about the referral recipient. Once the patient completes the form, the patient may submit the completed referral form to the referral platform. The referral platform may notify the referral recipient of the referral. The referral recipient may then access the referral platform to view the referred patient's information and follow up with the patient.

Aspects of the present disclosure may provide various technical advantages. For example, the referral platform may provide referral recipients with an integrated, customizable, and scalable way to receive, track, and manage referrals. By customizing or updating a referral form for each referral source, the referral recipient may tailor the links, input fields, and other options that are provided to patients based on who is referring the patients. Furthermore, because the smart cards may initially be agnostic as to referral source and recipient, and because the referral platform is configured to map one referral source with a plurality of referral sources- and with a customized referral form for each mapped referral source—the referral recipient may dynamically allocate smart cards to referral sources by determining a quantity of smart cards to synch with each referral source. Thus, by using data encoded on the smart card, the referral source may simply provide a smart card to a mobile device of a patient to refer the patient, thereby minimizing the amount of effort or organization required by the referral source to successfully refer the patient.

Furthermore, by scanning or tapping a card with a mobile device, the patient may receive a referral form from the referral platform and may complete the form even before leaving a consultation with a referral source, thereby increasing the likelihood that the patient successfully contacts a specialist in a timely manner and improving upon the speed and efficiency of some traditional computer techniques for facilitating electronic referrals. As a result of aspects of the present disclosure, not only is the referral process performed more quickly and accurately, but by reducing communication errors, the healthcare industry- or other industries that make use of referrals—may become more efficient and less costly.

Furthermore, by capturing patient action in real time, the referral platform may provide analytics data to referral recipients or referral sources. As a result, a referral recipient, for example, may have an integrated tool that includes data for patients referred to the referral recipient, that is communicatively coupled with other systems used by the referral recipient, and that provides analytics information for monitoring and improving the referral process. As will be apparent, these are only some of the advantages provided by aspects of the present disclosure.

Aspects of the present disclosure are described as being implemented in the healthcare industry. However, as will be understood by those of skill in the art, principles of the present disclosure are not limited to the healthcare industry. For example, aspects of the present disclosure may be implemented in other referral environments, such as industries involving services, law, accounting, manufacturing, technology, brokerage, banking and mortgage services, real estate, or business more generally.

### BRIEF DESCRIPTION OF THE DRAWINGS

FIG. 1 illustrates a network environment in which aspects of the present disclosure may be implemented.

FIG. 2 illustrates a communication diagram including the referral recipient, referral platform, and patient of FIG. 1.

FIG. 3 illustrates a schematic block diagram of an example architecture of the referral platform of FIG. 1.

FIG. 4 is a flowchart of an example method that may be performed by the referral recipient of FIG. 1.

FIG. 5. is a flowchart of an example method that may be performed by the referral platform of FIG. 1.

FIG. 6 is a flowchart of an example method that may be performed by the patient of FIG. 1.

FIG. 7 is a flowchart of an example method that may be performed by the referrer of FIG. 1.

FIG. 8 illustrates an example user interface of the referral platform of FIG. 1.

FIG. 9 illustrates an example user interface of the referral platform of FIG. 1.

FIG. 10 illustrates an example user interface of the referral platform of FIG. 1.

FIG. 11 illustrates an example user interface of a mobile application described herein.

FIG. 12 illustrates an example user interface displaying a referral form.

FIG. 13 illustrates a block diagram of an example computing system.

### DETAILED DESCRIPTION

Certain embodiments illustrated and described herein are only examples of the present technology. For example, although a referral recipient is described as a person, an entity that receives referrals may be a plurality of people, a professional group, an organization, an office, a computer system, or another entity type. Additionally, although a referrer or a referral source is described as a person, an entity that makes a referral may be a plurality of people, a professional group, an organization, an office, a computer system, or another entity type. Additionally, although described as a patient, an entity that is referred may be a plurality of people, a professional group, an organization, an office, a computer system, or another entity type. Furthermore, as described above, although aspects of the present disclosure are described in the context of the healthcare industry, the present disclosure is not limited to such a context. For example, aspects of the present disclosure may be implemented in other referral environments, such as industries involving services, law, accounting, manufacturing, technology, brokerage, banking, and mortgage services, real estate, or business more generally. Although aspects of the present disclosure are described in connection with the use of physical smart cards to facilitate referrals, the present disclosure is not limited to embodiments in which physical smart cards are used. For example, a virtual smart card or a software program that stores a URL may be used instead of a physical smart card. In other words, in some embodiments, a referrer may provide a link to a referral platform associated with a referral recipient by using other technology than a smart card that may encode or otherwise include a web address.

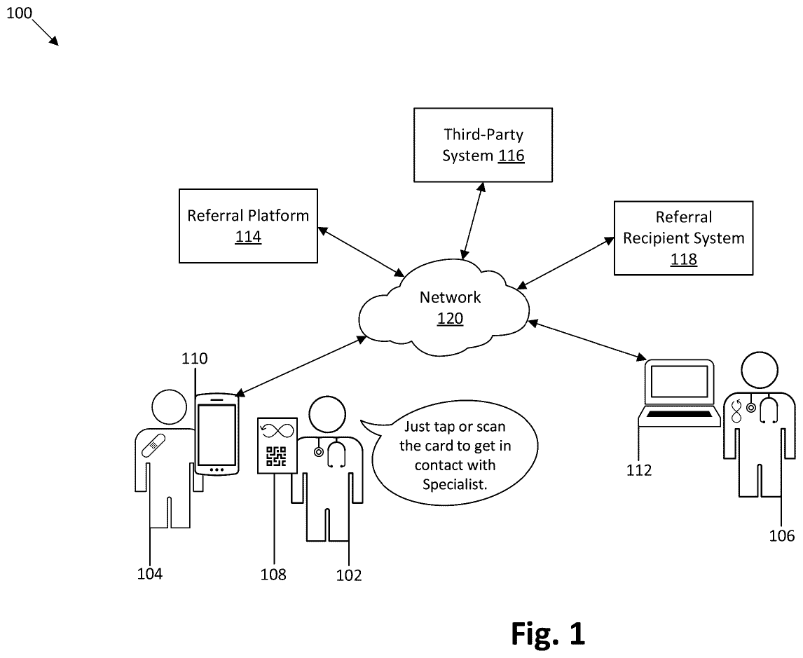

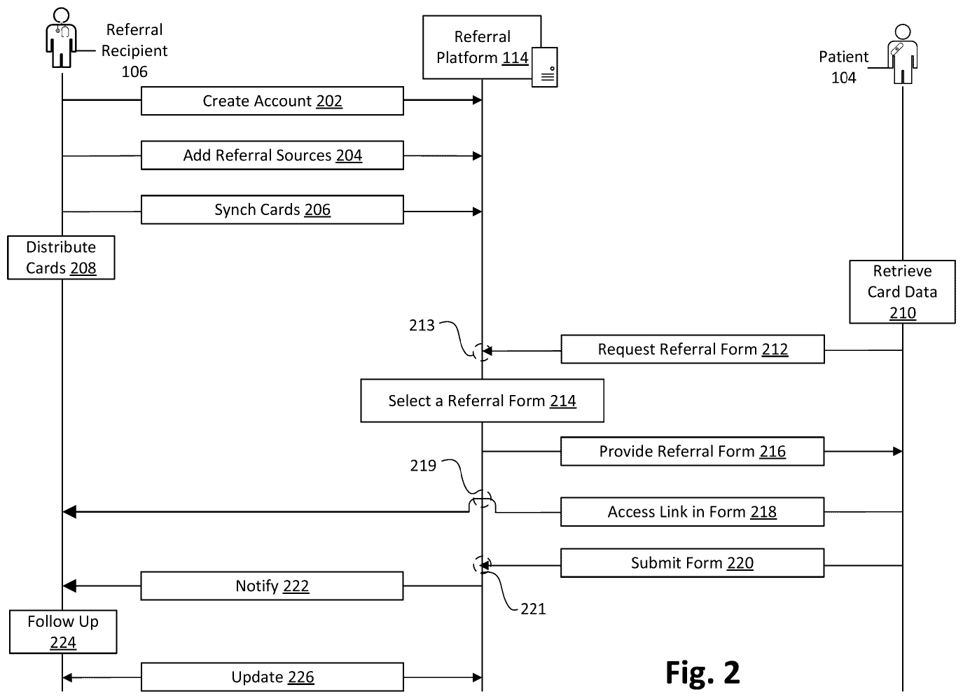

FIG. 1 illustrates a network environment 100 in which aspects of the present disclosure may be implemented. The environment 100 includes a referrer 102, a patient 104, a referral recipient 106, a card 108, devices 110-112, a referral platform 114, a third-party system 116, a referral recipient system 118, and a network 120. In some embodiments, the environment 100 may include more or fewer components than those in the example of FIG. 1. As a representative example application of the present disclosure, components of the environment 100 are described in the context of the healthcare industry. However, as described above, the present disclosure is not limited to the healthcare industry. Example operations of components of the environment are illustrated and described below in connection with FIG. 2.

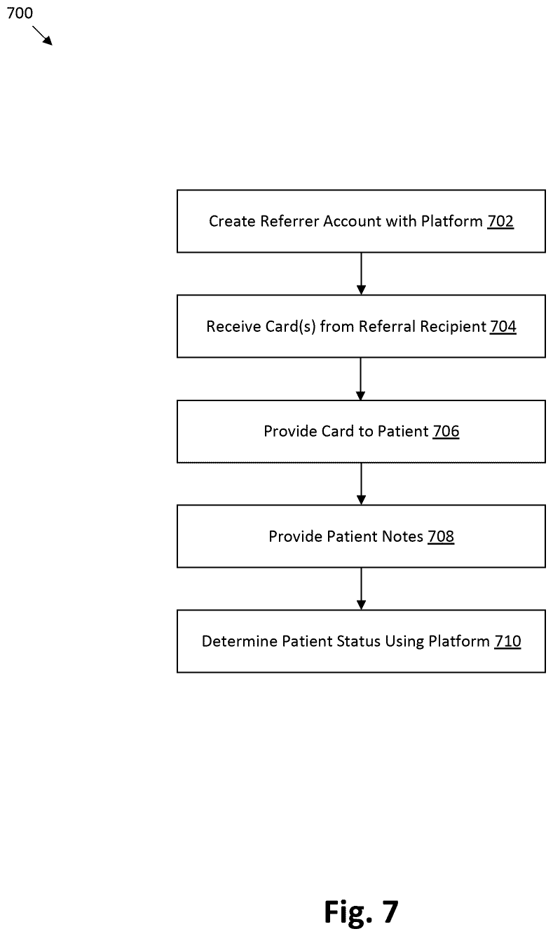

The referrer 102 may be a person that makes a referral (e.g., a “referral source”). For example, the referrer 102 may refer the patient 104 to the referral recipient 106. In some embodiments, the referrer 102 is a healthcare provider or healthcare professional that makes referrals. For example, the referrer 102 may be a dentist or general practitioner, or an office of dentists or general practitioners. The referrer 102 may possess the card 108, which the referrer 102 may use to refer patients to the referral recipient 106, a process that is further described below. In some embodiments, the referrer 102 may be a computer program. In some embodiments, the referrer 102 may use a device for connecting to the network 120 and communicating, for example, with the referral platform 114 or the referral recipient 106. For instance, in some embodiments, the referrer 102 may create an account with the referral platform 114. In some embodiments, the referrer 102 may provide notes to the referral recipient about the patient 104. Although illustrated as a single entity in the example of FIG. 1, there may be a plurality of referrers. For example, there may be a plurality of people having a card, such as a card similar to the card 108, that may be used to refer people to the referral recipient 106. An example method that may be performed by the referrer 102 is illustrated and described below in connection with FIG. 7.

The patient 104 may be a person that is referred by the referrer 102 to the referral recipient 106. Although referred to as a patient in the healthcare industry, the patient 104 may, in some embodiments, be more generally a person that is referred. In some instances, the patient 104 may physically go to see the referrer 102. In some instances, the patient 104 may remotely visit the referrer 102 (e.g., via a remote consultation). The referrer 102, after interacting with the patient 104, may decide to refer the patient to the referral recipient 106. To do so, the referrer 102 may provide the card 108 to the patient 104. As shown in the example of FIG. 1, the patient 104 may have a device 110 that may retrieve data from the card 108 and communicate with the referral platform 114. In some embodiments, the card 108 is a URL encoder that takes a different form from a physical smart card. For example, the card 108 may be a virtual card or another object that can be encoded to include readable data associated with a URL. Example operations that may be performed by the patient are illustrated and described below in connection with the FIGS. 2 and 7.

The referral recipient 106 may be a person that receives a referral from the referrer 102, such as a referral of the patient 104. In some embodiments, the referral recipient 106 is a healthcare specialist, such as an orthodontist or a surgeon. The referral recipient 106 may have an account with the referral platform 114 and may receive data from the patient 104 via the referral platform 114. Furthermore, the referral recipient 106 may receive and synch a plurality of cards, such as the card 108, such that when the patient 104 retrieves data from the card 108, the patient may retrieve data associated with the referral recipient 106 at the referral platform 114. In some embodiments, the referral recipient 106 may have a device 112 for connecting with the network 120. In some embodiments, the referral recipient 106 may be associated with the referral recipient system 118.

The card 108 may be a smart card that stores data. In some embodiments, the card 108 is a virtual smart card that may be stored, for example, on a device of the referrer 102. In some embodiments, the card 108 may store data corresponding to a Uniform Resource Locator (URL). The URL may, for example, lead to a web address associated with the referral platform 114. In some embodiments, the URL may include one or more parameters that the referral platform 114 links with data corresponding to the referral recipient 106 or the referrer 102. In some embodiments, each card of all cards that are associated with the referral platform 114 includes a unique identifier that is provided to and identified by the referral platform 114. In addition to storing a URL, the card 108 may also store other data, such as data related to the referral recipient 106, the referrer 102 or the referral platform 114.

The mobile device 110 may read data from the card 108. For example, the mobile device 110 may retrieve data related to one or more of the referral recipient 106, the referrer 102, or the referral platform 114 by reading the card 108. In some embodiments, the card 108 may include one or more of a QR code or near-field communication (NFC chip), and the mobile device 110 may read the card data using one or more of the QR code or the NFC chip. In some embodiments, the card 108 may store data using a different mechanism. The card 108 may include printed thereon a logo associated with the referral recipient 106. In some embodiments, the referral recipient 106 may receive the card 108, synch the card 108, and provide the card 108 to the referrer 102, which are processes that are further described below. The card 108 may be one of a plurality of cards associated with the referral recipient 106. Other cards of the plurality of cards associated with the referral recipient 106 may be possessed by referral sources other than the referrer 102.

The device 110 may be a device that can read data from the card 108 and that can access the referral platform 114. In examples, the device 110 is a mobile phone or other smart device. However, the device 110 may also be another computing device, such as a laptop. In some embodiments, the device 110 may include one or more of an NFC reader or a camera. In some embodiments, the device 110 may include a web browser for interacting with the referral platform 114. In some embodiments, the device 110 may execute software that, in response to receiving data from the card 108 (e.g., after scanning a QR code of the card 108 or using an NFC reader to extract data from an NFC chip of the card 108), generates a selectable notification on a display of the device 110. When the notification is selected, the device 110 may use the browser to access the referral platform 114 using data retrieved from the card 108. In some embodiments, the device 110 may use a mobile application to access the referral platform 114.

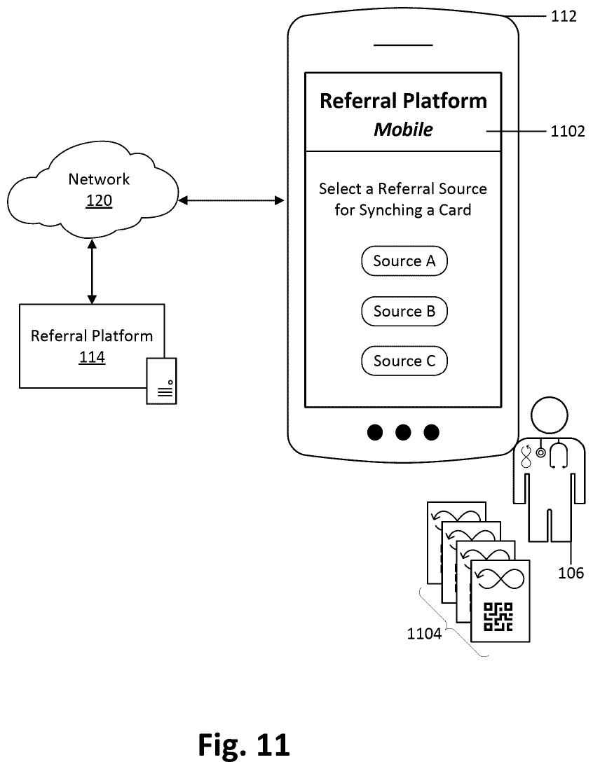

The device 112 may be a computing device that may communicate with the referral platform 114. For example, as illustrated in the example of FIG. 1, the device 112 may be a computer. However, the device 112 may be a mobile phone or another computing device, as illustrated, for example, by the device 112 of FIG. 11. The device 112 may use one or more of a web browser or mobile application to communicate with the referral platform 114.

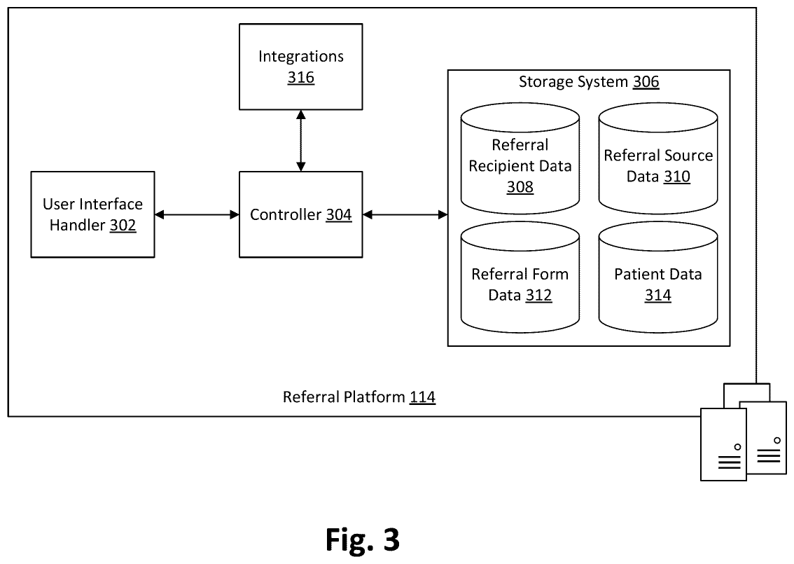

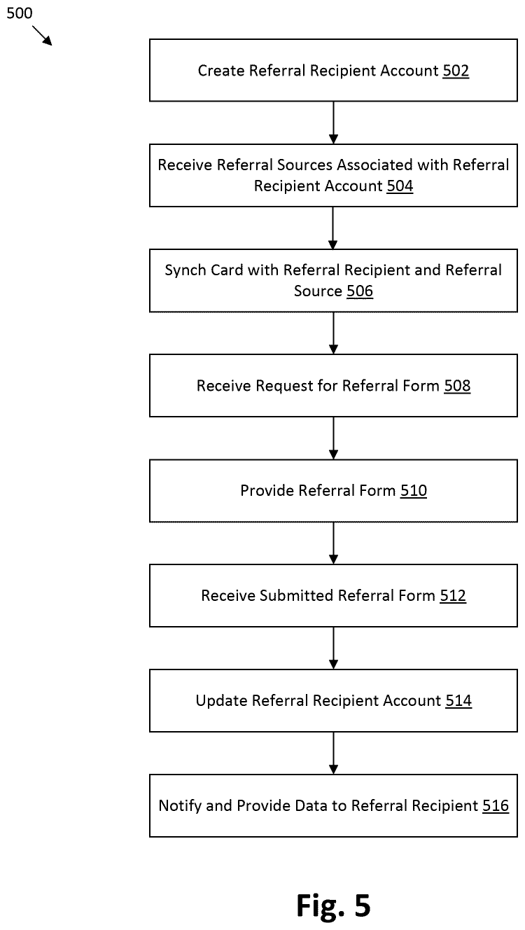

The referral platform 114 may, among other things, facilitate the making, receiving, and tracking of referrals. The referral platform 114 may have a plurality of users. For example, there may be a plurality of people that have accounts with the referral platform and that receive referrals using the referral platform 114, such as the referral recipient 106. Furthermore, the referral platform 114 may provide a referral form to the patient 104, receive data from the patient 104, and provide the data to the referral recipient 106. In some embodiments, the referrer 102 may have an account with the referral platform 114 or otherwise interact with the referral platform 114. Example operations of the referral platform 114 are illustrated and described below in connection with FIGS. 2 and 5. An example architecture of the referral platform 114 is illustrated and described below in connection with FIG. 3.

The third-party system 116 may be a system that interacts with one or more other components of the environment 100. In some embodiments, the third-party system 116 may be a website or a system that provides software as a service. In some embodiments, the patient 104 may access the third-party system 116 by clicking a link in a referral form displayed by the device 110. Although illustrated as a single entity in the example of FIG. 1, the environment 100 may include a plurality of third-party systems 116. Example third-party systems include a social media site, an email server, an online scheduling system, an insurance-related service, or another system. In some embodiments, the referral platform 114 may automatically exchange data with the third-party system 116 by using application programming interface (API) integrations, as described further in connection with FIG. 3. In some embodiments, the third-party system 116 includes a third-party software service.

The referral recipient system 118 may be a system that is associated with the referral recipient 106 or an organization associated with the referral recipient 106. In some embodiments, the referral recipient system 118 may be a type of system that a third-party system may be (e.g., an online scheduling system, an insurance-related service, a virtual consultation system, etc.), except the referral recipient system 118 may be associated with the referral recipient 106. In examples, the referral recipient system 118 may be one or more of a website of the referral recipient 106.

The network 120 may be, for example, a wireless network, a wired network, a virtual network, the internet, or another type of network. The network 120 may be divided into subnetworks, and the subnetworks may be different types of networks or the same type of network. In different embodiments, the network environment 100 can include a different network configuration than shown in FIG. 1, and the network environment 100 may include more or fewer components than those illustrated.

FIG. 2 illustrates a communication diagram illustrating example data exchanges and operations of aspects of the present disclosure. The example of FIG. 2 illustrates the referral recipient 106, the referral platform 114, and the patient 104, each of which are illustrated in FIG. 1.

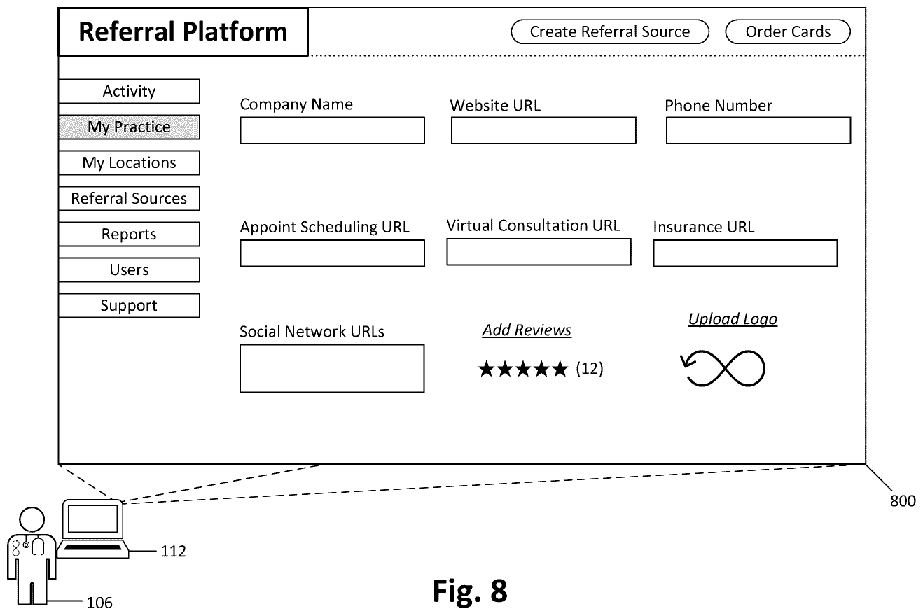

At operation 202, the referral recipient 106 may create an account with the referral platform 114. In some embodiments, the referral recipient may access a web page of the referral platform 114 for creating an account. In some embodiments, the account may be specifically for users who are seeking to receive referrals using the referral platform 114. As part of creating the account, the referral recipient may provide various types of registration data to the referral platform 114. For example, the referral recipient 106 may provide a name, a description, a logo, location data, certification data, payment information, security information (e.g., passwords or keys), authorized user information, one or more links to system or services, or other data. An example user interface via which the referral recipient 106 may create an account is illustrated and described below in connection with FIG. 8.

The location data may include addresses, descriptions, reviews, or other information for a plurality of locations associated with the referral recipient 106. For example, if the referral recipient 106 is a healthcare specialist or a group of healthcare specialists, then the referral recipient 106 may provide location data for each of the locations at which the healthcare specialist or group of healthcare specialists work.

The one or more links to systems or services may be URLs that lead to a system or service associated with the referral recipient 106. As examples, the one or more links may include a URL for accessing an online scheduling system of the referral recipient, information regarding insurance accepted by the referral recipient, a web site of the referral recipient, a social media profile of the referral recipient, a reviews page of the referral recipient, or another web resource.

The authorized user information may include data for one or more users who have authorization to access an account of the referral recipient 106. For example, the referral recipient 106 may grant access to an employee to view referral data on the referral platform 114. As another example, the referral recipient 106 may grant access to one or more referral sources, so that the referral sources may communicate directly with the referral recipient 106 via the referral platform 114. In some embodiments, granting access to a referral source may include creating an account with limited access that can be used by the referral source to communicate with the referral recipient 106 and view data associated with patients referred by the referral source. In some embodiments (e.g., if the referral recipient is an office of people that receive referrals), there may be a plurality of authorized users having separate profiles with the referral platform 114.

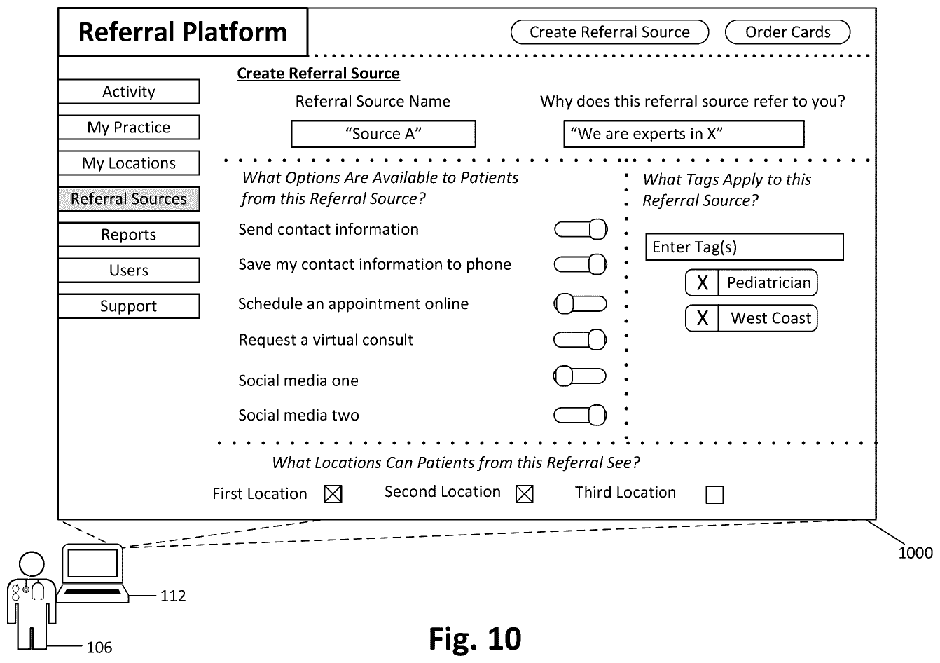

At operation 204, the referral recipient 106 may add referral sources. As an example, if the referral recipient is a healthcare specialist, such as an orthodontist, the referral recipient 106 may know a number of referral sources (e.g., dentists) from whom the referral recipient 106 has received referrals in the past or from whom the referral recipient 106 would like to receive referrals. For each of these referral sources, the referral recipient may add a profile with the referral platform. To do so, the referral recipient 106 may login into his or her account and navigate to a page for adding a referral source. The referral recipient 106 may then input data for the referral source (e.g., name, practice type, location, contact information, etc.). The referral recipient 106 may also customize a referral form that the referral source's patients will see when referred to referral recipient 106. The customization may include defining input fields, adding and customizing links to web resources, or selectively displaying information. The referral recipient 106 may also add one or more customized tags that will apply to that referral source. The referral recipient 106 may then save the referral source with the referral platform 114 and then proceed to add another referral source. An example user interface for adding a referral source is illustrated in the example of FIG. 10.

In some embodiments, as part of adding referral sources, the referral recipient 106 may create accounts for referral sources on the referral platform 114. For example, for a given referral source, the referral recipient 106 may select an option to create a referrer account on the referral platform 114. In response, the referral platform 114 may send a message to the referral source to access his or her account and finalize its creation. Using this account, the referral source may use a web application of the referral platform 114 to perform one or more actions, such as tracking patient progress for the patient's referred by the referral source or communicating with the referral recipient. Furthermore, in some embodiments, the referral source may use the account to refer new or existing patients to the referral recipient 106. As such, the referral source may have the option of using a smart card to refer a patient using the referral source (as described further herein), or the referral source may directly use a web application of the referral platform 114 to refer a patient without using a patient's device or a smart card.

At operation 206, the referral recipient 106 may synch cards, such as the card 108 of FIG. 1. For example, the referral recipient 106 may order smart cards from the referral platform 114. When received by the referral recipient 106, the smart cards may be blank (e.g., they may not be linked to any particular referral recipient or referral source). In some embodiments, however, the smart cards may be generally linked to the referral platform 114. Synching the cards may include associating each card with the referral recipient and with one of a plurality of referral sources. As an example, if a referral recipient 106 created referral source profiles for Referral Source A, Referral Source B, and Referral Source C, the referral recipient 106 may synch 10 cards with Referral Source A, 7 cards with Referral Source B, and 3 cards with Referral Source C. Advantageously, because the smart cards are initially blank, the referral recipient 106 may dynamically allocate cards to existing referral sources or allocate them to new referral sources.

To synch a card, the referral recipient 106 may, in some embodiments, use a mobile application associated referral platform 114. For example, the referral recipient 106 may use a mobile device to download and execute a mobile application associated with the referral platform 114. The mobile application may be configured to allow the referral recipient to log into his or her account associated with the referral platform 114. The mobile application may then present a plurality of referral sources with which the referral recipient 106 is associated. For each card, the referral recipient 106 may select one of the plurality of referral sources. Then the referral recipient 106 may use a mobile device to interact with the card to synch the card with the selected referral source. For example, the mobile device may read data from an NFC chip in the smart card. As another example, the mobile device may scan a QR code of the smart card. In some embodiments, the user may perform separate operations for synching the NFC chip and the QR code. In some embodiments, a different data transfer technology other than an NFC chip or QR code is used.

Continuing with the synching operation, the mobile application, which may be communicatively coupled with other components of the referral platform 114, may have data related to the referral recipient 106 (e.g., because the referral recipient is logged into the mobile application), the selected referral source, and the card. Thus, the referral platform 114 may store a mapping between the referral recipient, the referral source, and the synched card. Thus, when a patient uses the card to access the referral platform 114, the referral platform 114 may provide data related to the referral recipient 106 and the referral source for which the card was synched. For example, the referral platform 114 may provide, to the patient, the referral form that the referral recipient 106 customized for patients of the referral source. In a similar manner, the referral recipient 106 may synch each card of a plurality of cards.

In some embodiments, synching a smart card causes data encoded on the smart code to be associated with a particular referral recipient account and referral source. For example, due to a synching operation, the smart code may include an identifier (e.g., an alphanumeric code) that is associated with the referral recipient account and the referral source. In some embodiments, this identifier may be two separate identifiers. In some embodiments, this identifier is part of a URL that is encoded on the smart code.

At operation 208, the referral recipient 106 may distribute synched smart cards. For example, the referral recipient 106 may, for each smart card of a plurality of synched smart cards, distribute the smart card to the referral source with which the smart card was synched. For example, referring to the example above, the referral recipient 106 may provide 10 smart cards synched with Referral Source A to the Referral Source A, 7 smart cards synched with the Referral Source B to the Referral Source B, and 3 smart cards synched with the Referral Source C to the Referral Source C.

At operation 210, the patient may retrieve card data. For example, the patient 104 may use a mobile device to retrieve data encoded on a smart card provided to the patient 104 by a referral source (e.g., a referral source to which the referral recipient 106 distributed the smart card). Depending on the embodiment, patient 104 may tap the card with the mobile device (e.g., thereby reading data from an NFC chip of the smart card using an NFC reader of the mobile device) or scan the smart card using a camera of the mobile device. In some embodiments, the card data retrieved by the patient 104 may include a URL associated with the referral platform 114. In some embodiments, the mobile device may, after retrieving data from the card, display a notification asking the patient 104 whether the patient would like to access the URL with a browser of the mobile device. In some embodiments, the patient 104 may use the browser of the mobile device to follow the URL. In some embodiments, the data retrieved from the card may be useable by a mobile application installed on the patient's mobile device, and the mobile application may be used to access aspects of the referral platform 114.

At operation 212, the patient may request a referral form from the referral platform 114. Requesting the referral form may include using the URL read from the smart card to access the referral platform 114.

At operation 213, the referral platform 114 may detect that the patient 104 requested a referral form. The referral platform 114 may log this activity. Referring generally to the operations 213, 219, and 221, the referral platform 114 may monitor and log user interaction (e.g., patient interaction) with the referral platform 114. The referral platform 114 may log the type of action performed by the user (e.g., requested a referral form, submitted a referral form, followed a link in a referral form, etc.), a timestamp, and an identifier of the user or one or more identifiers of entities with which the user is associated (e.g., a referral recipient to which a patient is referred or a referral source that referred the patient). In some embodiments, the referral platform 114 may use logged activity to provide analytics data to one or more of the referral recipient 106 or a referral source. In some embodiments, the referral platform 114 may update the analytics data in real time as new events are captured. In some embodiments, the referral platform may be communicatively coupled with a plurality of mobile devices. In such embodiments, the referral platform 114 may use one or more of load balancing or parallel processing techniques. For example, the referral platform 114 may simultaneously process requests from a plurality of different mobile devices that retrieved data from different smart cards. Furthermore, each of the of the different smart cards may have previously been synched by the referral platform and may be associated with different referral sources.

At operation 214, the referral platform 114 may select a referral form. For example, the referral platform 114 may select the referral form associated with the smart card tapped or scanned by the user. The smart card may be associated with the referral recipient 106 and the referrer that provided the smart card to the patient 104. Thus, the referral platform 114 may select the referral form that is customized for the referral recipient 106 and the referrer that provided the smart card to the patient 104.

At operation 216, the referral platform 114 may provide the selected referral form to the patient 104. For example, if accessed via a web browser, the referral platform 114 may provide the selected referral form to the web browser. If accessed via a mobile application, the referral platform 114 may provide the selected referral form to the mobile application. The patient's mobile device may display the selected referral form. The patient may then begin to fill out the referral form or interact with links, data, or other options of the referral form. In some embodiments, any data that is part of the referral for or that is input using the referral form may be considered referral data.

At operation 218, the patient may access a link in the referral form. As described above in connection with the operations 202-204, the referral recipient 106 may provide links that are to be provided to patients referred from referral sources. As examples, the links may be for scheduling systems, virtual consultation resources, a website associated with the referral recipient 106, social media profiles, reviews, insurance information, contact information, or other resources. When the patient 104 selects one of these links, then the mobile device (e.g., the browser installed thereon) may access the resource corresponding to the selected link. Although the operation 218 is illustrated in the example of FIG. 2 as leading to the referral recipient 106, patient 104 may, depending on the selected link, access a third-party system. In some embodiments, the patient 104 may access a plurality of links of the referral form. For instance, the patient 104 could access a link to view insurance information and then access a link to schedule an appointment with the referral recipient 106. In some embodiments, the links may also be a deep link to a mobile application installed on a mobile device of the patient 104. In some embodiments, a link, when selected, may cause the mobile device of the patient 104 to email, text, or call the referral recipient 106.

At operation 219, the referral platform 114 may detect that the patient 104 selected a link or option in the referral form. Thus, even if the patient 104 is accessing a third-party system or is not accessing other aspects of the referral platform 114, the referral platform 114 may nevertheless capture activity of the patient 104 and include that activity in analytics provided to the referral recipient 106.

At operation 220, the patient 104 may submit the form. For example, having filled out one or more input fields of the referral form, the patient may select an option to submit the completed referral form to the referral platform 114. In some embodiments, the completed referral form need not have all data fields of the referral form to be filled to be a completed referral form. Example fields of the referral form for which the patient 104 may provide data include, but are not limited to, the following: a name, a message, contact information, available contact times, an email, a phone number, a preferred communication medium, an indication of whether the patient 104 would like to be contacted, available appointment times, health information of the patient 104, or other referral data. The referral platform 114 may determine whether the patient 104 has provided data for all required fields, if there are any required fields. The referral platform 114 may receive the submitted the referral form and extract the data provided by the patient 104. In some embodiments, the referral platform 114 may provide a confirmation or a follow-up form to the patient 104. In some embodiments, the referral platform 104 may automatically send an alert (e.g., an email, text, or other type of alert) to the referral recipient 106 that a referral has been received.

At operation 221, the referral platform 114 may log activity of the patient 104 when the patient 104 submits the referral form.

At operation 222, the referral platform may notify the referral recipient 106 of the referral of the patient. For example, the referral platform 114 may notify the referral recipient 106 that the patient 104 submitted data to the referral platform 114 by filling out the referral form. In some embodiments, to notify the referral recipient 106, the referral platform 114 may automatically cause an email to be sent to the referral recipient 106. In some embodiments, the referral platform 114 may text, send a push notification, or notify the referral recipient in a different way. In some embodiments, the referral platform 114 may notify a system used by the referral recipient to manage referrals or schedule consultations. In some embodiments, the referral platform 114 may not send the patient data to the referral recipient 106 along with the notification. Instead, to access the patient data, the referral recipient 106 may be required to log into an account with the referral platform 114. In some embodiments, the referral platform 114 may update data associated with referral recipient 106 indicating that the referral recipient has a patient to follow up with.

At operation 224, the referral recipient 106 may follow up with the patient 104. For example, the referral recipient 106 may access the referral platform 114 to view information provided by the patient 104 and to view other data related to the patient (e.g., patient activity or notes about the patient provided by the referral source). Based on the information, the referral recipient 106 may contact the patient 104. In some embodiments, the referral recipient 106 may call, text, email, or otherwise contact the patient. In some embodiments, the referral recipient 106 may use a software program to contact the patient.

At operation 226, the referral recipient 106 may update the referral platform 114. For example, the referral recipient 106 may indicate that the referral recipient 106 has followed up with the patient 104. In response, the referral platform 114 may update data related outstanding referrals and provide the updated data to referral recipient. In some embodiments (e.g., when the referral platform 114 is integrated with systems used by the referral recipient 106), systems used by the referral recipient 106 may automatically update the referral platform 114 in response to the referral recipient 106 contacting, or attempting to contact, the patient 104.

FIG. 3 illustrates a block diagram of example components of the referral platform 114. The referral platform 114 may be a combination of software and hardware and may be implemented by using various computing systems and environments. The referral platform 114 may include both frontend and backend components. In some embodiments, the referral platform 114 may be implemented using a model-view-controller framework. In some embodiments, aspects of the referral platform 114 may be implemented using Laravel and Filament. In some embodiments, the referral platform 114 may be implemented in a cloud-based environment. In the example shown, the referral platform includes a user interface handler 302, a controller 304, a storage system 306, and integrations 316. In some embodiments, one or more of the components illustrated in FIG. 3 may be implemented using one or more servers.

The user interface handler 302 may manage aspects of frontend applications of the referral platform 114. For example, the user interface handler 302 may manage a plurality of user interfaces. The user interface handler 302 may manage the displaying and receiving of data via the user interfaces. The user interfaces may belong to a website associated with the referral platform 114. The user interfaces may include, but are not limited to, a user interface for a referral recipient to create an account, add referral sources, manage locations, manage authorized users, or perform other operations. Additionally, there may be a user interface for presenting and analyzing data, which may include one or more data visualizations that may be updated in real time. The user interface handler 302 may also be responsible for referral forms that are provided to patients. Additionally, the user interface handler 302 may be responsible for user interfaces of a mobile application associated with the referral platform 114 that may be used, for example, to synch smart cards with referral recipients and referral sources. In some embodiments, the user interface handler 302 may also receive requests from users of the referral platform 114 and may provide the requests to the controller 304. In some embodiments, the user interface handler 302, or the controller 304, may expose an endpoint (e.g., one or more APIs) of the referral platform 114 that may be accessed by users to communicate with the referral platform 114.

The controller 304 may be communicatively coupled with the user interface handler 302 and the storage system 306. The controller 304 may receive requests from users and may execute software to respond to the requests. In some instances, the controller 304 may retrieve data from the storage system 306 and provide the retrieved data to the user (e.g., via the user interface handler 302). In some instances, the controller 304 may store data in the storage system 306 based on information received from a user of the referral platform 114. In some embodiments, the controller 304 may execute, using other aspects of the referral platform 114 or other systems or programs, operations described herein as being performed by the referral platform 114.

The storage system 306 may include one or more databases that store data received by the referral platform 114 or generated by the referral platform 114. In the example shown, the storage system includes referral recipient data 308, referral source data 310, referral form data 312, and patient data 314. The data components illustrated in the example of FIG. 3 may be stored in a common database or separate databases. In some embodiments, the storage system 306 may use a MySQL database to store at least some of the data 308-3114. In some embodiments, the storage system 306 may include more or less data than described in the example of FIG. 3. In some embodiments, databases of the storage system 306 requires that data have a certain format. For example, the databases may require that data have a particular type, field, or file format. As such, in some embodiments, the referral platform 114 may convert referral data received from the mobile device (which may not be compatible with databases of the storage system 306) into a format that can be ingested by the storage system 306.

The referral recipient data 308 may include data related to users who receive referrals via the referral platform 114, such as the referral recipient 106. The referral recipient data 308 may include account information for referral recipients. For example, the referral recipient data 308 may include data input by a user when creating an account. Furthermore, the referral recipient data 308 may include subscription data, payment information, and other data related to a referral recipient or a referral recipient account. Furthermore, the referral recipient data 308 may include data related to the smart cards and referral sources with which the referral recipient is associated.

The referral source data 310 may include data related to referral sources. For example, the referral source data 310 may include data input by a referral recipient when creating a referral source. Furthermore, in some embodiments, a referral source may create an account with the referral platform 114, and the referral source data 310 may include data for the referral source account. The referral source data 310 may further include data related to activity of referral sources, such as a number of referrals made by a referral source, a number of patients from the referral source who became patients of the referral recipient, or other data derived from activity of a referral source or of a patient of a referral source.

The referral form data 312 may include data related to the referral forms provided to patients. In some embodiments, the referral form data may include customized templates for a plurality of referral forms. For example, based on data received from the referral recipient 106, the referral platform 114 may define a referral form for a particular referral source that is associated with the referral recipient account. These referral forms may in part be customized and may be an incomplete template provided to patients and referrers to be completed. Each customized template may be associated with a unique pairing of a referral recipient and a referral source and may be further associated with one or more smart cards synched for the referral recipient and the referral source.

The patient data 314 may include data for patients. The patient data 314 may be from submitted referral forms that include data input by a patient when completing a referral form. Furthermore, the patient data 314 may include data related to patient activity, such as events caused by a patient submitting a referral form, following a link in a referral form, contacting the referral recipient, or any other activity that may be detected by the referral platform 114.

The integrations 316 may include one or more APIs for facilitating data exchanges with external systems. For example, the integration 316 may enable the referral platform 114 to provide data to third-party software services that may be used by the referral recipient 106. Such systems may include, for example, software that performs scheduling tasks on behalf of the referral recipient, that creates and manages patient charts, that performs data entry tasks, or that performs other tasks. In some embodiments, the software is practice management software. In an example implementation, when the referral platform 114 receives a referral, the controller 304 may extract data from that referral and automatically provide such data to external systems by using an API of the integrations 316. For example, using the integrations 316, the controller 304 may provide a patient name, email, phone number, and referral source to a scheduling system (or other system) via the integrations 316, and then the referral recipient 106 may access such data via those systems. In some embodiments, the integrations may enable bidirectional communication between the referral platform 114 and the external systems. For example, the referral platform 114 may receive data from the external systems, such as for example, scheduling data, chart data, or other data, depending on the properties of the external system.

In some embodiments, the referral data received by the referral platform 114 may have a first format (e.g., a format associated with the referral form). In some embodiments, the first format may not be compatible with a third-party software service via which the referral platform 114 is integrated. For example, the third-party software service may require certain data types, data fields, or file formats. As such, in response to receiving a completed referral form having referral data, the referral platform 104 may, in some embodiments, automatically convert the referral data into a format that can be ingested by the third-party software service.

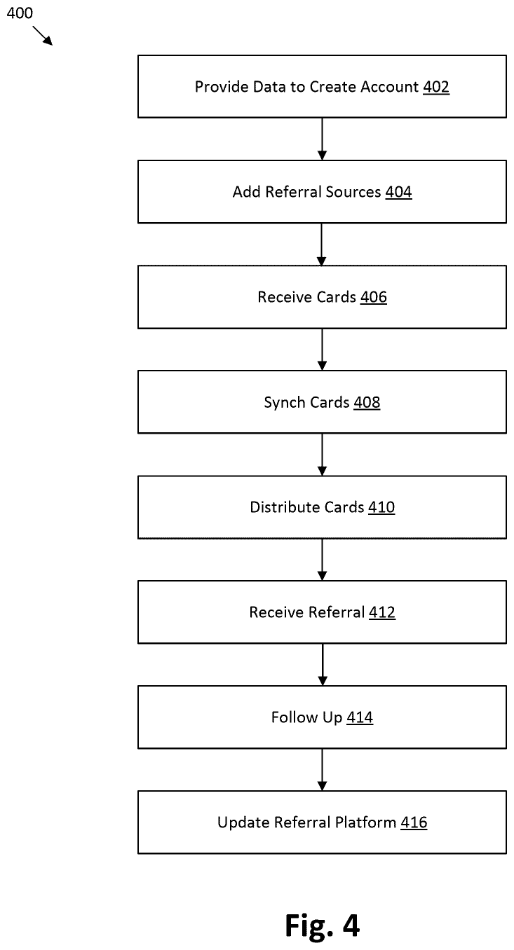

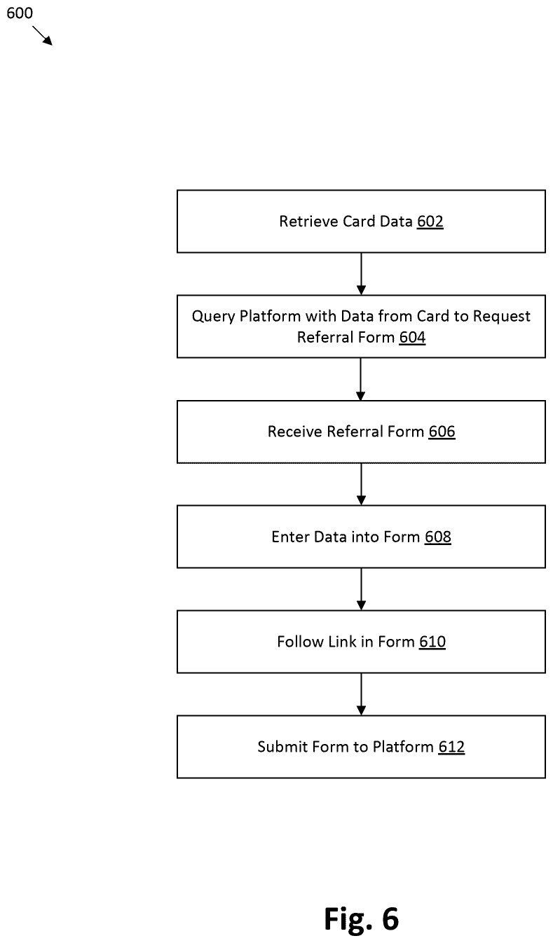

FIGS. 4-7 illustrate flowcharts of example methods that may be performed by aspects of the present disclosure. As examples, the method of FIG. 4 may be performed by the referral recipient 106, the method of FIG. 5 may be performed by the referral platform 114, the method of FIG. 6 may be performed by the patient 104, and the method of FIG. 7 may be performed by the referrer 102. However, one or more of the methods or operations described in connection with the FIGS. 4-7 may be performed by a different entity than as described in the subsequent discussion of FIGS. 4-7.

FIG. 4 is a flowchart of an example method 400. In the following description, the method 400 is described as performed by the referral recipient 106. However, in some embodiments, one or more operations of the method 400 may be performed by another entity, such as a different component of the environment 100.

At operation 402, the referral recipient 106 may provide data to the referral platform 114 to create an account. An example of creating an account is described above in connection with the step 202 of FIG. 2.

At operation 404, the referral recipient 106 may add referral sources. An example of adding referral sources is described above in connection with the operation 204 of FIG. 2.

At operations 406, the referral recipient 106 may receive cards. For example, the referral recipient 106 may order cards using the referral platform 114. In some embodiments, the referral platform 114 may offer a plurality of smart card types that may be ordered by the referral recipient 106. The plurality of smart card types may include smart cards made of different materials (e.g., plastic, metal, or a mixture of materials). In some embodiments, the referral platform 114 may offer smart cards that include different data communication technology, such as smart cards that include QR codes, smart cards that use NFC chips, smart cards that include both a QR code and an NFC chip, or smart cards that implement different data transfer technology.

At operation 408, the referral recipient 106 may synch cards. An example of synching cards is described above in connection with the operation 206 of FIG. 2.

At operation 410, the referral recipient 106 may distribute cards. An example of distributing cards is described above in connection with the operation 208 of FIG. 2.

At operation 412, the referral recipient 106 may receive a referral, such as a referral of the patient 104. For example, the referral platform 114 may notify the referral recipient 106 that the referral recipient has received a referral, as described above in connection with the operation 222 of FIG. 2. The referral recipient 106 may, in some embodiments, access an account of the referral platform 114 to view information related to the referral, which may include data input by the patient 104, data input by the referrer 102 (e.g., notes about the patient 104), metadata about the referral (e.g., a time, location, referral type, etc.), or other data.

At operation 414, the referral recipient 106 may follow up with the patient 104. An example of following up with the patient is described above in connection with the operation 224 of FIG. 2.

At operation 416, the referral recipient 106 may update the referral platform 114. For example, once the referral recipient 106 has followed up with the patient 104, the referral recipient 106 may send data to the referral platform 114 indicating that the referral recipient 106 has followed up with the patient 104. Furthermore, in some embodiments, the referral recipient 106 may provide additional data regarding patient 104, such as whether the patient scheduled a consultation, a result of the consultation, notes about the patient, a message to the referrer 102 about the patient 104, or other data. In some embodiments, a system or service used by the referral recipient 106 to follow up with patients will automatically provide an update to the referral platform 114 in response to the referral recipient 106 using the system or service to follow up with the patient 104.

FIG. 5 is a flowchart of an example method 500. In the following description, the method 500 is described as performed by the referral platform 114. However, in some embodiments, one or more operations of the method 500 may be performed by another entity, such as a different component of the environment 100. Additionally, the referral platform 114 may perform other operations that those described in connection with the method 500.

At operation 502, the referral platform 114 may create an account for a referral recipient. For example, the referral recipient may provide data to the referral platform (e.g., as described above in connection with the step 202 of FIG. 2), and the referral platform 114 may, in response to receiving the data, create or update an account for the referral recipient. For example, the referral platform 114 may store data in the referral recipient data 308 of FIG. 3 as part of creating a referral recipient account.

At operation 504, the referral platform 114 may receive referral sources associated with a referral recipient account. For example, for a given referral recipient, the referral platform 114 may receive data for a plurality of referral sources associated with the referral recipient, as described above in connection with the operation 204 of FIG. 2. In examples, the referral platform 114 may store the referral source data in the referral source data 310. Additionally, for each referral source associated with a given referral recipient, the referral platform 114 may store a referral form based on data input by the referral recipient for that referral source. In examples, the referral form may be stored in the referral form data 312 and may be mapped to the referral recipient and the referral source.

At operation 506, the referral platform 114 may synch a smart card with a referral recipient and a referral source. For example, the referral platform 114 may receive, via a mobile application, data from a user that is synching a smart card. The data may include an identifier for a referral recipient, a referral source with which the referral recipient is associated, and the smart card itself. In some embodiments, data that identifies the smart card may include a URL for accessing the referral platform 114. Having received the data, the referral platform 114 may associate the referral recipient and referral source identifier with the data related to the smart card. Furthermore, the referral platform 114 may associate the referral form that is mapped to the referral recipient and the referral source with the data related to the smart card. In some embodiments, for a given pair of referral recipient and referral source, the referral platform 114 may perform multiple synch operations, such as a synch operation for each of a plurality of data transfer technologies of the smart card (e.g., a separate synch process for a QR code and an NFC chip). In some embodiments, the referral platform 114 may repeat the operation 506 for a plurality of smart cards.

At operation 508, the referral platform 114 may receive a request for a referral form. For example, a user, such as a patient, may access a website of the referral platform 114 by using data retrieved from a previously synched smart card. The patient's browser may use a unique URL that has been associated with a particular referral recipient and referral source. An example of receiving a request for a referral form and selecting the appropriate referral form is described above in connection with the operations 212-214 of FIG. 2

At operation 510, the referral platform 114 may provide the patient's browser with a referral form that is customized for that referral recipient and referral source. An example of providing a referral form is described above in connection with the FIG. 216 of FIG. 2

At operation 512, the referral platform 114 may receive a submitted referral form, an example of which is described above in connection with the operation 220 of FIG. 2.

At operation 514, the referral platform 114 may update a referral recipient account to reflect the newly received referral. For example, the referral platform 114 may update a number that indicates how many patients the referral recipient must follow up with, and the referral platform 114 may update data that is used in an analytics page.

At operation 516, the referral platform 114 may notify the referral recipient of the referral, a process that is described above in connection with the operation 222 of FIG. 2. Furthermore, the referral platform 114 may provide data to the referral recipient regarding the patient (e.g., in response to the referral recipient accessing his or her account on the referral platform 114). Additionally, referral platform 114 may receive data updates from the referral recipient once the referral recipient has followed up with the referral.

FIG. 6 is a flowchart of an example method 600. In the following description, the method 600 is described as performed by patient 104. However, in some embodiments, one or more operations of the method 600 may be performed by another entity, such as a different component of the environment 100. Additionally, the patient 104 may perform more or fewer operations that those described in connection with the method 600.

At operation 602, the patient 104 may retrieve card data. An example of retrieving card data is described above in connection with the operation 210 of FIG. 6.

At operation 604, the patient 104 may query the referral platform 114 using data retrieved from the smart card to request a referral form. An example of requesting a referral form is described above in connection with the operation 212 of FIG. 2.

At operation 606, the patient 104 may receive a referral form. For example, the patient 104 may receive, using a device used to retrieve data from the smart card and to query the platform, a referral form that is provided by the referral platform 114. An example of the referral platform 114 providing the referral form to the patient 104 is described above in connection with the operation 216 of FIG. 2.

At operation 608, the patient 104 may enter data into the form. For example, the patient 104 may enter data into one or more input fields displayed by the referral form. Depending on the embodiment, the patient 104 may type, use a touch screen, or use voice input to enter data into the form. In some embodiments, the patient 104 may be required to fill out one or more input fields, whereas other input fields may be optional.

At operation 610, the patient 104 may follow a link in the referral form, a process that is described above in connection with the operation 218 of FIG. 2. In some embodiments, the patient 104 may then return to a display of the referral form and may continue, or begin, to fill out the referral form.

At operation 612, the patient 104 may submit the referral form to the referral platform 114, a process that is further described above in connection with the operation 220 of FIG. 2. Then the patient 104 may, in some instances, receive a follow up from the person to which the patient 104 was referred. In some instances, however the patient 104 may use a link in the referral form to directly schedule an appointment with the person to which the patient was referred.

FIG. 7 is a flowchart of an example method 700. In the following description, the method 700 is described as performed by referrer 102. However, in some embodiments, one or more operations of the method 700 may be performed by another entity, such as a different component of the environment 100. Additionally, the referrer 102 may perform more or fewer operations that those described in connection with the method 700.

At operation 702, the referrer 102 may create an account with the referral platform 114. In some embodiments, the account may be for a person or office that makes referrals (e.g., a referral source), and such an account may have a different configuration than an account made for a referral recipient. In some embodiments, the referrer 102 may provide data related to his or her practice, locations, times, patients or referrals, people to whom the referrer 102 makes referrals (e.g., referral recipients), or other data. In some embodiments, the referrer account may be for a plurality of people, such as an account for an office of general practitioners or dentists.

At operation 704, the referrer 102 may receive one or more cards from a referral recipient. As described herein, the referrer 102 may use the cards to refer patients to the referral recipient. In some embodiments, the referrer 102 may receive multiple cards form the same referral recipient, so that multiple people in the office can refer patients to the referral recipient or so that there are replacement cards in case if a card is lost or damaged. In some embodiments, the referrer 102 may receive cards from a plurality of referral recipients.

At operation 706, the referrer 102 may provide a card to a patient to refer the patient to a referral recipient associated with the card.

At operation 708, the referrer 102 may provide notes about a referred patient. In some embodiments, the referrer 102 may use an account on referral platform 114 to provide notes about the patient. In some embodiments, the referrer 102 may provide notes about the patient to the referral platform by using the referral form that is submitted by the patient. For example, the referrer 102 may use a microphone of the mobile device 110 to input audio data, such as a voice message, that will be provided to the referral platform 114 and accessed by the referral recipient 106. In some embodiments, the referral recipient may view notes entered by the referrer 102 by using the referral platform 114. In some embodiments, the referrer 102 may provide notes directly to the referral recipient (e.g., via email or another communication medium).

At operation 710, the referrer 102 may determine a status of a patient using the platform 114. For example, the referrer 102 may use an account to view data related to a patient referred by the referrer 102. In some embodiments, the referrer 102 may view whether the referral recipient followed up with the patient, whether the patient received the requisite care, notes input by the referral recipient regarding the patient, or other data related to the patient.

Referring generally to the FIGS. 8-10, user interfaces that may be part of a frontend application of the referral platform 114 are illustrated. In the examples shown, the referral recipient 106 may navigate to the user interfaces illustrated in FIGS. 8-10 by accessing a website of the referral platform 114. As shown, the website may include a plurality of selectable pages, such as “Activity,” “My Practice,” “My Locations,” and so on. Each of the pages may include different displays for showing data to the users of the website and different input fields for receiving data from users. In addition to the example pages illustrated, the user interfaces may also include displays or options, such as the buttons shown for creating a referral source and ordering cards. Additionally, although the FIGS. 8-10 are described in the context of a website, the illustrated user interfaces, data, and functionality may, in some embodiments, be part of a mobile application.

FIG. 8 illustrates an example user interface 800 for a “My Practice” page of a website associated with the referral platform 114. As shown, the user interface 800 includes a plurality of input fields via which the referral recipient 106 may provide data related to his or her practice. In some embodiments, aspects of the user interface 800 may be used to create or update a referral recipient account. Additionally, in some embodiments, data input into the user interface 800 may be included in a referral form provided to patients.

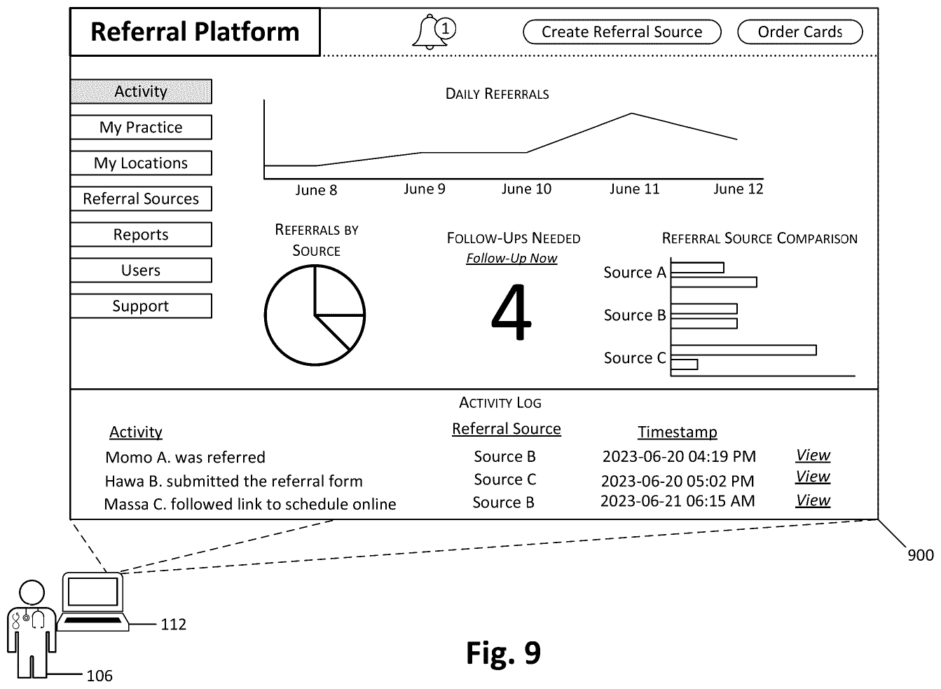

FIG. 9 illustrates an example user interface 900 for an “Activity” page of a website associated with the referral platform 114. The user interface 900 may include data related to a practice of the referral recipient 106, to patients referred to the referral recipient 106, to referral sources associated with the referral recipient 106, to data related to the referral platform 114, or to other data. As shown, the user interface 900 may include analytics data that may be updated in real time. For example, the user interface 900 may include charts, graphs, and data tables. For example, the user interface 900 may illustrate a number of referrals received by the referral recipient 106 by day, a number of referrals received by referral source, a number of patients with whom the referral recipient 106 needs to follow up, and a conversion rate of referrals broken down by referral source (e.g., a proportion of referrals with whom the referral recipient 106 ends up having an appointment). In some embodiments, the referral recipient 106 may filter the analytics data by using the custom-defined tags described in connection with FIG. 10.

Additionally, the user interface 900 includes a patient activity log that displays patient activity. The activity log may include, for example, data captured by the referral platform 114 as patients interact with a referral form. In some embodiments, the activity log may include one or more of the following: a type of activity, a patient name, a referral source associated with the patient, a timestamp, a current status (e.g., awaiting a follow up, currently scheduled, etc.), and a link to view more information about the patient (e.g., notes about the patient or data input by the patient). In other examples, the user interface 900 may include more or fewer components than described in connection with FIG. 9.

Additionally, the user interface 900 includes an alert feature, as shown, for example, by the bell indicating that there is one pending alert. The referral platform 114 may generate alerts in response to various conditions. In some embodiments, the alerts may relate to configuring an account for the referral recipient 106. For example, if the referral recipient 106 has not yet taken an action to set up or complete an account, then the referral platform 114 may generate an alert to notify the referral recipient 106. As another example, the referral platform 114 may analyze data associated with the referrals received by the referral recipient 106 and by the practice of the referral recipient 106 more generally. If the referral platform 114 detects one or more of a trend, anomaly, area for opportunity, or other relevant condition, the referral platform 114 may generate an alert. In some embodiments, the referral platform 114 may use a machine learning model trained on one or more of data associated with the referral recipient 106 or other referral recipients to generate alerts. For example, the machine learning model may be trained to perform an inference task using data collected by the referral platform 114. In some embodiments, the referral recipient 106 may also provide instructions or resources to the referral recipient 106 regarding how to respond to the alert. In some embodiments, instead of (or in addition to) providing an alert via the user interface 900, the referral platform 104 may send an alert to the referral recipient 106 via one or more of an email, text, push notification, or message via another system that is used by the referral recipient 106.

FIG. 10 illustrates an example user interface 1000 for a “Referral Sources” page of a website associated with the referral platform 114 In the example shown, the user interface 1000 includes input fields for creating a referral source. In the example shown, the referral recipient 106 is adding “Source A,” which may be a person, office, or system that provides referrals to the referral recipient 106. Using aspects of the user interface 1000, the referral recipient 106 may configure settings for Source A. For example, the referral recipient 106 may customize a referral form for patients that are referred by Source A. For example, the referral recipient may select which of a plurality of options are available to patients referred by Source A and which locations are visible to patients referred by Source A.

In some embodiments, the referral form received by patients referred to the referral recipient 106 by Source A may include the options and locations enabled by the referral recipient 106 via the user interface 1000. On the other hand, patients may not be able to use or see options or locations that are disabled by the referral recipient 106 via the user interface 1000. In some embodiments, a display for a Referral Sources page may include a listing of existing referral sources for the referral recipient 106. In some embodiments, the referral recipient 106 may selectively enable and disable referral sources. For instance, in some embodiments, only referral sources that are enabled by the referral recipient 106 may be able to refer patients to the referral recipient 106 by using smart cards and the referral platform 114.

Continuing with the example of FIG. 10, the user interface 1000 includes a region for applying keyword tags to a referral source. A tag may be a custom-defined category of data. For example, the referral recipient 106 may create a “Pediatrician” tag. When creating a referral source, the referral recipient 106 may apply the “Pediatrician” tag to the referral source if that referral source is, in fact, a pediatrician, and the referral recipient 106 may use the “Pediatrician” for a plurality of other referral sources. Then when analyzing data, the referral recipient may use the custom-defined tags as filters. For example, the referral recipient 106 may view referrals, statistics, visualizations, or other data associated with referral sources that have been tagged with the “Pediatrician” tag. In this way, the referral recipient 106 may customize how data is analyzed and displayed, thereby enabling the referral recipient 106 to derive insights and take actions based on activity data associated with a group of entities defined by the referral recipient 106.

In some embodiments, different users of the referral platform 114 may create different tags. For example, a first specialist may create a first set of tags that are relevant to the first specialist, and a second specialist may create a second set of tags that are relevant to the second specialist. In some embodiments, a plurality of tags may be applied to a single referral source (e.g., both “Pediatrician” and “West Coast,” as shown). Example tags may include, but are not limited to, the following: practice type; geographical area, such as city, state, zip code, region, or country; school district; or other category of interest to the referral recipient.

The user interfaces illustrated in the examples of FIGS. 8-10 are examples of only some possible user interfaces and user interface configurations of the referral platform 114.

For example, the referral platform 114 may include a user interface for displaying or generating reports. The reports may include, for example, referral recipient data, patient data, referral source data, or analytics data. In some embodiments, a reports user interface may include one or more visualization that allow a user to view referrals by referral source and to view referrals over time. Furthermore, the reports may include visualizations and other data that are filtered based on customized tags. In some embodiments, the reports may be exported (e.g., as a CSV, XLSX, PDF, DOCX, or other type of file). Furthermore, in some embodiments, the referral recipient 106 may share a report with another use of the referral platform 114.

As another example, the referral platform 114 may include a user interface for managing existing referral sources. For example, the referral platform 114 may include a user interface that includes a display region for each of a plurality of existing referral sources. The user interface may include input fields for editing data of an existing referral source, generating a report for an existing referral source, or toggling an activation status of an existing referral source. For example, are referral source may be deactivated, which may, in some instances, disable the ability of the deactivated referral source from making referrals using the referral platform 114 or the smart cards. By having the ability to toggle between an activated and deactivated state for referral sources, the referral recipient 106 may, in some embodiments, easily control (e.g., without deleting or recreating a referral source account) which referral sources may send recommendations and when.

As another example, the referral platform 114 may also include a user interface for managing locations, which may include address information, contact information for a location, data about services provided at a location, and other location-specific information. As another example, the referral platform 114 may include a user interface for managing users. For example, one account may be associated with a plurality of users, each of whom may have a different level of access. In some embodiments, an administrator of the account may set an access level for each of the other authorized users of an account. As another example, the referral platform 114 may include a user interface for receiving technical support from an administrator of the referral platform 114 and to manage a subscription with the referral platform 114.

As another example, the referral platform 114 may include a user interface for reviewing activity for an individual patient. Such activity may display the patient's visit and communication history. In some embodiments, the user interface may indicate a current stage for interacting with that patient. For example, the user interface may indicate that a first appointment has been scheduled for that patient or that the patient has not yet been contacted.

FIG. 11 illustrates a schematic diagram of the referral platform 114 communicatively coupled with the device 112 via the network 120. As illustrated by the example of FIG. 11, the referral recipient 106 may use the device 112 to synch the cards 1104 with the referral platform 114. An example of the referral recipient 1106 synching the cards with the referral platform 114 is described above in connection with the operation 206 of FIG. 2

In the example of FIG. 11, the device 112 is a mobile device with a mobile application 1102 installed thereon. The mobile application 1102 includes a user interface that the referral recipient 106 may use as part of synching the cards 1104 with the referral platform 114. In some embodiments, the mobile application 1102 may be available for download in a mobile app store.

As an example of using the mobile application 1102 to synch the plurality of cards 1104, the referral recipient 106 may select, for each card of the plurality of cards 1104, a referral source with which to synch the card by using the mobile application 1102. For example, the referral recipient 106 may select Source A, Source B, or Source C, each of which may have previously been added by the referral recipient to the referral platform 114. Prior to being synched, each of the plurality of cards 1104 may be blank.

Once the referral recipient 106 selects a referral source (e.g., “Source B”), then the mobile application 1102 may request that the referral recipient scan or tap the selected card. The mobile application 1102 may then provide data retrieved from the card, data related to the referral recipient 106 (e.g., as determined by a profile on the mobile application 1102), and data related to the selected referral source to the referral platform 114, which may map the selected card with the referral recipient 106 and the selected referral source. In some embodiments, the referral recipient 106 may perform multiple synching operations for each card, and the referral recipient 106 may synch each of the plurality of cards 1104. An example of a synched card of the plurality of cards 1104 is the card 108 of FIG. 1. In some embodiments, a different technique may be used to synch one or more of the cards 1104.

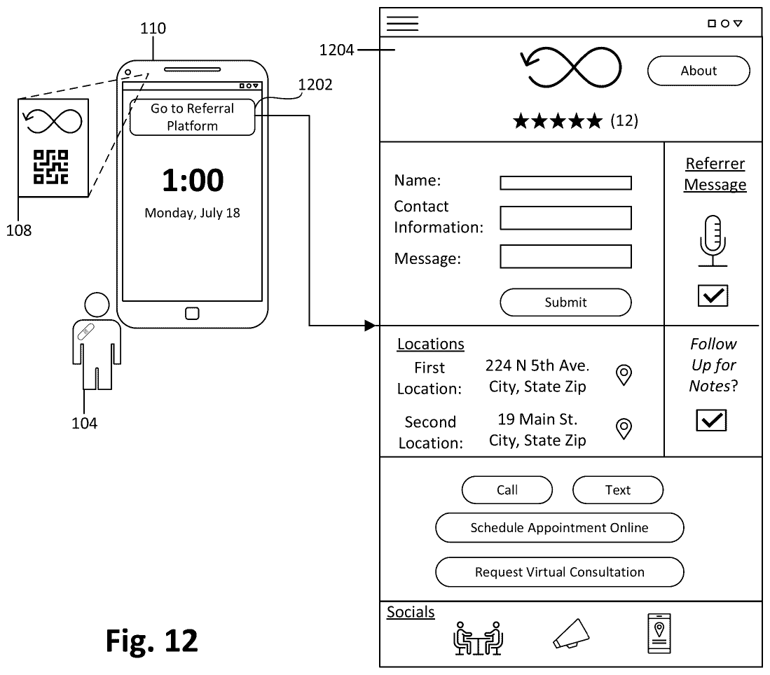

FIG. 12 illustrates a schematic diagram of the patient 104 and the mobile device 110. As shown in the example of FIG. 12, the patient 104 may use the mobile device 110 to retrieve data from the card 108, as described above in connection with the operation 210 of FIG. 2. The card 108 may be associated with someone to whom the patient 104 is being referred (e.g., the referral recipient 106). In response to retrieving data form the card 108, the mobile device 110 may display a notification 1202 to the patient 104. In some embodiments, the notification 1202 may be selected by the patient 104. In response to receiving a selection of the notification 1202, the mobile device 110 may activate a browser and navigate to an aspect of the referral platform 114 to request a referral form, which may be provided by the referral platform 114 to the mobile device 110.

In the example shown, the referral form 1204 may be displayed via a browser of the mobile device 110. In the example shown, the referral form 1204 includes information about a referral recipient, such as a logo, reviews, and a link to more information. Furthermore, the referral form 1204 includes a plurality of input fields via which the patient 104 may input information. Furthermore, the referral form 1204 includes locations of the referral recipient, including addresses and selectable fields that may open a navigation mobile application. Furthermore, the referral form 1204 includes a plurality of buttons, which may be links to other systems. In the example of FIG. 12, the referral form 1204 includes links to schedule an appointment online, request a virtual consultation, and navigate to one or more social media profiles of the referral recipient. The referral form 1204 may also include other links, such as links to insurance information, to general information, to related practitioners, or to other systems, website, or applications. In some embodiments, the links may include one or more web links or one or one or more links to mobile applications. Additionally, as shown by the example of FIG. 12, the referral form 1204 may include options to contact the referral recipient (e.g., by calling, texting, or emailing).

As shown in the example of FIG. 12, the referral form 1204 may include components for a referral source (e.g., the referrer 102) to submit a message. The message may be, for example, notes about the patient 104, such as a reason for which the patient 104 is being referred, a diagnosis of the patient 104, or other information. Such a message may provide important information to the referral recipient so that the referral recipient can appropriately interact with or treat the patient 104. In some embodiments, the referrer may input a message by submitting text into an input field of the referral form 1204. In some embodiments, the message left by the referral source may be submitted to the referral platform 114 along with other data of the referral form 1204, and the referral recipient 106 may access the message by using the referral form 1204.

In some embodiments, the referrer may record a voice message using a microphone of the mobile device 110. For example, the referrer may select a microphone icon (or other input field) of the referral form 1204 to begin recording a voice message, and once the message is recorded, a completion indication may be displayed (e.g., a checkmark). With respect to the voice message feature, the web application displaying the referral form 1204 may, in some embodiments, use an API offered by a browser of the mobile device 110. The API may enable the web application to access a microphone of the mobile device 110 that can record audio data. In some embodiments, the referral form may be provided by a native mobile application, which may be configured to access a microphone of the mobile device 110. The voice message feature may enable the referrer to use a device of the patient to input a voice message, which is then provided to servers of the referral platform 114, where it can be accessed by the referral recipient 106. This arrangement may result in better outcomes for all parties. For example, it may result in a higher likelihood that the referrer leaves a message, and the message may have more detail than messages transmitted via other means.

In some embodiments, the referral form 1204 includes an input field that may be selected by the patient or the referrer about following up for notes. If this input field is selected, then the referral platform 114 may instruct the referral recipient to follow up with the referrer for notes regarding the patient 104. For example, if the referrer wants to provide a note to the referral recipient but, for whatever reason, is unable to do so while consulting with the patient 104, this input field may be selected. As a result, the referral recipient may then contact the referrer at a later time so that the referrer can provide notes to the referral recipient. Advantageously, as shown the by the example of FIG. 12, aspects of the present disclosure provide numerous channels via which the referral source and referral recipient may communicate with one another, including via a text message, a voice message, or an instruction to follow up later. Furthermore, the referral source and the referral recipient may be able to communicate using a web application of the referral platform when the referral source uses a portal on the referral platform 114 or an account set up by the referral recipient.

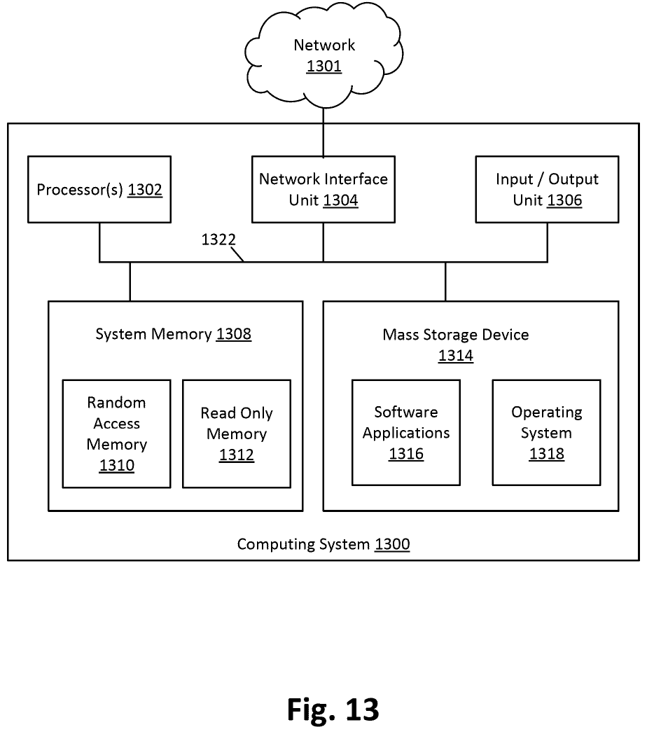

FIG. 13 illustrates an example block diagram of a virtual or physical computing system 1300. One or more aspects of the computing system 1300 can be used to implement the system and processes described herein.

In the embodiment shown, the computing system 1300 includes one or more processors 1302, a system memory 1308, and a system bus 1322 that couples the system memory 1308 to the one or more processors 1302. The system memory 1308 includes RAM (Random Access Memory) 1310 and ROM (Read-Only Memory) 1312. A basic input/output system that contains the basic routines that help to transfer information between elements within the computing system 1300, such as during startup, is stored in the ROM 1312. The computing system 1300 further includes a mass storage device 1314. The mass storage device 1314 is able to store software instructions and data. The one or more processors 1302 can be one or more central processing units or other processors.

The mass storage device 1314 is connected to the one or more processors 1302 through a mass storage controller (not shown) connected to the system bus 1322. The mass storage device 1314 and its associated computer-readable data storage media provide non-volatile, non-transitory storage for the computing system 1300. Although the description of computer-readable data storage media contained herein refers to a mass storage device, such as a hard disk or solid-state disk, it should be appreciated by those skilled in the art that computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, DVD (Digital Versatile Discs), other optical storage media, magnetic cassettes, magnetic tape, magnetic disk storage or other magnetic storage devices, or any other medium which can be used to store the desired information and which can be accessed by the computing system 1300.

According to various embodiments of the invention, the computing system 1300 may operate in a networked environment using logical connections to remote network devices through the network 1301. The network 1301 is a computer network, such as an enterprise intranet and/or the Internet. The network 1301 can include a LAN, a Wide Area Network (WAN), the Internet, wireless transmission mediums, wired transmission mediums, other networks, and combinations thereof. The computing system 1300 may connect to the network 1301 through a network interface unit 1304 connected to the system bus 1322. It should be appreciated that the network interface unit 1304 may also be utilized to connect to other types of networks and remote computing systems. The computing system 1300 also includes an input/output controller 1306 for receiving and processing input from a number of other devices, including a touch user interface display screen, or another type of input device. Similarly, the input/output controller 1306 may provide output to a touch user interface display screen or other type of output device.

As noted above, the mass storage device 1314 and the RAM 1310 of the computing system 1300 can store software instructions and data. The software instructions include an operating system 1318 suitable for controlling the operation of the computing system 1300. The mass storage device 1314 and/or the RAM 1310 also store software instructions, that when executed by the one or more processors 1302, cause one or more of the systems, devices, or components described herein to provide functionality described herein. For example, the mass storage device 1314 and/or the RAM 1310 can store software instructions that, when executed by the one or more processors 1302, cause the computing system 1300 to receive and execute managing network access control and build system processes.

While particular uses of the technology have been illustrated and discussed above, the disclosed technology can be used with a variety of data structures and processes in accordance with many examples of the technology. The above discussion is not meant to suggest that the disclosed technology is only suitable for implementation with the data structures shown and described above.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., operations, memory arrangements, etc.) described with respect to the figures herein are not intended to limit the technology to the particular aspects described. Accordingly, additional configurations can be used to practice the technology herein and/or some aspects described can be excluded without departing from the methods and systems disclosed herein.

Similarly, where operations of a process are disclosed, those operations are described for purposes of illustrating the present technology and are not intended to limit the disclosure to a particular sequence of operations. For example, the operations can be performed in differing order, two or more operations can be performed concurrently, additional operations can be performed, and disclosed operations can be excluded without departing from the present disclosure. Further, each operation can be accomplished via one or more sub-operations. The disclosed processes can be repeated. Furthermore, as used herein, when an operation is performed at a component, this indicates that the component or one or more of its subcomponents may perform the operation.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

What is claimed is:

1. A method for facilitating referrals, the method comprising:
at a referral platform, creating a referral recipient account and a referral source associated with the referral recipient account;
at the referral platform, defining a referral form for the referral source associated with the referral recipient account;
at the referral platform, synching a smart card with the referral source and the referral recipient account, wherein the synched smart card encodes a uniform resource locator (URL) and an identifier associated with the referral recipient account and the referral source, wherein the URL includes a web address of the referral platform;
at a mobile device, tapping or scanning the smart card to read the URL and the identifier from the smart card;
at the mobile device, using the URL and the identifier, transmitting a request to the referral platform for the referral form;
at the referral platform, in response to receiving the request, identifying the referral form using the identifier;
at the referral platform, providing the referral form to the mobile device;
at the mobile device, receiving referral data to complete the referral form, the referral data including text data input by a patient via a touch screen of the mobile device and audio data recorded by a microphone of the mobile device, the audio data being input by a healthcare provider;
at the mobile device, providing the completed referral form to the referral platform; and
at the referral platform, receiving the completed referral form and providing the completed referral form to a referral recipient associated with the referral recipient account.

2. The method of claim 1, further comprising, after receiving the completed referral form, automatically performing, at the referral platform:
converting the referral data of the referral form from a first format incompatible with a third-party software service to a second format compatible with the third-party software service;
providing, via an application programming interface (API) integration, the referral data to the third-party software service;
converting the referral data into a third format compatible with a data storage system of the referral platform;
updating the data storage system of the referral platform to store the referral data and create a mapping between the referral data, the referral source, and the referral recipient account;
generating a message containing at least some of the referral data; and
providing the message to the referral recipient by sending one or more of a push notification or email to a computing device of the referral recipient.

3. The method of claim 1, wherein the smart card includes a quick response (QR) code or a near field communication (NFC) chip, the QR code or the NFC chip encoding the URL and the identifier.

4. The method of claim 1,
wherein synching the smart card with the referral source and the referral recipient account comprises using a mobile application associated with the referral platform;
wherein the mobile application is installed on a computing device of the referral recipient;
wherein the referral recipient uses the mobile device to tap or scan the smart card; and
wherein, using the mobile application, the referral recipient selects the referral source, from among a plurality of referral sources, for synching the smart card.

5. The method of claim 1, further comprising:
at the referral platform, applying a customized tag to the referral source; and
at the referral platform, automatically filtering and storing the referral data based on the customized tag in response to identifying that the completed referral form is associated with the referral source;
wherein the customized tag identifies a practice type or region of the referral source.

6. The method of claim 1, further comprising:
at the referral platform, generating analytics data for the referral recipient account, the analytics data comprising an activity log;
at the referral platform, displaying the analytics data via a visualization; and
at the referral platform, automatically updating the visualization in response to receipt of the completed referral form.

7. The method of claim 1, wherein the identifier includes a first identifier associated with the referral recipient account and a second identifier associated with the referral source.

8. The method of claim 1, further comprising:
creating a plurality of referral sources associated with the referral recipient account, the plurality of referral sources including the referral source; and
synching a plurality of smart cards, wherein each smart card of the plurality of smart cards is synched with the referral recipient account and one referral source of the plurality of referral sources;
wherein each smart card of the plurality of smart cards includes the web address of the referral platform and each smart card includes a different identifier corresponding to a respective referral source with which each smart card is synched.

9. The method of claim 1, further comprising:
at the referral platform, analyzing referral recipient account data to identify a condition associated with one or more of a trend or anomaly;
at the referral platform, in response to identifying the condition, automatically generating an alert and providing the alert to the referral recipient.

10. The method of claim 9, wherein analyzing the referral recipient account data to identify the condition comprises performing an inference task with a machine learning model trained using data collected by the referral platform.

11. The method of claim 1, further comprising, at the referral platform, simultaneously processing requests from a plurality of different mobile devices that retrieved data from different smart cards, each of the different smart cards having previously been synched by the referral platform.

12. A system for facilitating referrals, the system comprising:
a referral platform comprising a server;
a mobile device; and
a network communicatively coupling the mobile device and the server of the referral platform;
wherein the server of the referral platform is configured to:
create a referral recipient account associated with a referral recipient and a referral source associated with the referral recipient account;
define a referral form for the referral source associated with the referral recipient account;
synch a URL encoder with the referral source and the referral recipient account, wherein the synched URL encoder encodes a uniform resource locator (URL) and a code identifying the referral recipient account and the referral source, the URL including a web address of the referral platform;
in response to receiving a request from the mobile device that includes the code, retrieve the referral form and provide the referral form to the mobile device;
receive a completed referral form from the mobile device and store the completed referral form;
wherein the mobile device is configured to:
tap or scan the URL encoder to read the URL and the code from the URL encoder;
using the URL and the code, transmit the request to the referral platform for the referral form;
receive, via one or more input devices of the mobile device, referral data to complete the referral form; and
provide the completed referral form to the referral platform.

13. The system of claim 12, wherein receiving, via the one or more input devices of the mobile device, the referral data to complete the referral form comprises receiving text data from a patient via touch screen and receiving audio data from a healthcare provide via a microphone.

14. The system of claim 12, wherein the referral platform is implemented in a cloud-based environment.

15. The system of claim 12, wherein the referral platform is further configured to, in response to receiving the completed referral form, automatically perform:
convert the referral data of the referral form from a first format incompatible with a third-party software service to a second format compatible with the third-party software service; and
provide, via an application programming interface (API) integration, the referral data to the third-party software service.

16. The system of claim 12, wherein the referral platform is further configured to, in response to receiving the completed referral form, automatically perform:
convert the referral data into a format compatible with a data storage system of the referral platform;
store the referral data in the data storage system;
generate a message containing at least some of the referral data; and
provide the message to the referral recipient by sending an email to the referral recipient.

17. The system of claim 12, wherein the URL encoder is a smart card that includes a quick response (QR) code or a near field communication (NFC) chip, the QR code or the NFC chip encoding the URL and the code.

18. The system of claim 12, wherein the referral platform is further configured to receive an update from a computing system of the referral recipient, the update indicating that the referral recipient contacted a patient that submitted the referral form.

19. The system of claim 18, wherein the referral platform is further configured to automatically update an analytics page in response to receiving the completed referral form and in response to receiving the update.

20. A platform for facilitating referrals, the platform comprising:
a processor; and
memory storing instructions;
wherein the platform is communicatively coupled with a mobile device; and
wherein the instructions, when executed by the processor, cause the platform to:
create a referral recipient account and a referral source associated with the referral recipient account;
define a referral form for the referral source associated with the referral recipient account;
synch a smart card with the referral source and the referral recipient account, wherein the synched smart card encodes a uniform resource locator (URL) and an identifier associated with the referral recipient account and the referral source, wherein the URL includes a web address of the referral platform;
in response to receiving a request from the mobile device after the mobile device taps or scans the smart card to retrieve the URL and the identifier, identify the referral form using the identifier;
provide the referral form to the mobile device;
receive a completed referral form from the mobile device, the mobile device having received referral data to complete the referral form, the referral data including text data input by a patient via a touch screen of the mobile device and audio data recorded by a microphone of the mobile device, the audio data being input by a healthcare provider;
provide the completed referral form to a referral recipient associated with the referral recipient account.
